(19) United States
(12) Patent Application Publication
(10) Pub. No.: US 2017/0132371 A1
(43) Pub. Date: May 11, 2017

(54) Automated Patient Chart Review System and Method

(71) Applicant: Parkland Center for Clinical Innovation, Dallas, TX (US)

(72) Inventors: Ruben Amarasingham, Dallas, TX (US); George Oliver, Southlake, TX (US); Timothy Scott Swanson, Grapevine, TX (US); Allison Gilley, Dallas, TX (US); Ellen Araj, Dallas, TX (US); Ying Ma, Southlake, TX (US); Paea LePendu, Dallas, TX (US); Yukun Chen, Dallas, TX (US); Nora Huri, Dallas, TX (US); Anand Shah, Dallas, TX (US)

(21) Appl. No.: 15/297,107

(22) Filed: Oct. 18, 2016

Related U.S. Application Data
(60) Provisional application No. 62/243,653, filed on Oct. 19, 2015.

(51) Int. Cl. G06F 19/00; G06F 17/21; G06F 17/30; G06F 17/27

(52) U.S. Cl. CPC G06F 19/322; G06F 19/328; G06F 19/3437; G06F 19/3431; G06F 17/271; G06F 17/21; G06F 17/277; G06F 17/2785; G06F 17/30867; G06F 17/30554; G06Q 50/01

(57) ABSTRACT

A computerized method of automated patient chart review includes receiving a selection of a particular patient, automatically parsing at least one document of a patient's medical record having structured data and natural language data, automatically generating a list of variables from the patient's medical record, automatically generating a list of important variables from the list of variables associated with a specific clinical event from the structured data and natural language data. Predictive modeling and artificial intelligence are used to analyze the patient data, reviewer actions, and reviewer feedback data.

### RELATED APPLICATION

This patent application claims the benefit of U.S. Provisional Patent Application No. 62/243,653 filed on Oct. 19, 2015 and is related to the following co-pending patent/patent applications, all of which are incorporated herein by reference:

U.S. Non-Provisional patent application Ser. No. 14/835,698 filed on Aug. 25, 2015, entitled “Clinical Dashboard User Interface System and Method,” now U.S. Pat. No. 9,147,041 issued on Sep. 29, 2015;

U.S. Non-Provisional patent application Ser. No. 14/798,630 filed on Jul. 14, 2015, entitled “Client Management Tool System and Method”;

U.S. Non-Provisional patent application Ser. No. 14/682,557 filed on Apr. 9, 2015, entitled “Holistic Hospital Patient Care and Management System and Method For Automated Resource Management”;

U.S. Non-Provisional patent application Ser. No. 14/682,610 filed on Apr. 9, 2015, entitled “Holistic Hospital Patient Care and Management System and Method For Patient and Family Engagement”;

U.S. Non-Provisional patent application Ser. No. 14/682,668 filed on Apr. 9, 2015, entitled “Holistic Hospital Patient Care and Management System and Method For Situation Analysis Simulation”;

U.S. Non-Provisional patent application Ser. No. 14/682,705 filed on Apr. 9, 2015, entitled “Holistic Hospital Patient Care and Management System and Method For Automated Staff Monitoring”;

U.S. Non-Provisional patent application Ser. No. 14/682,745 filed on Apr. 9, 2015, entitled “Holistic Hospital Patient Care and Management System and Method”;

U.S. Non-Provisional patent application Ser. No. 14/682,807 filed on Apr. 9, 2015, entitled “Holistic Hospital Patient Care and Management System and Method For Telemedicine”;

U.S. Non-Provisional patent application Ser. No. 14/682,836 filed on Apr. 9, 2015, entitled “Holistic Hospital Patient Care and Management System and Method For Automated Patient Monitoring”;

U.S. Non-Provisional patent application Ser. No. 14/682,866 filed on Apr. 9, 2015, entitled “Holistic Hospital Patient Care and Management System and Method For Enhanced Risk Stratification”;

U.S. Non-Provisional patent application Ser. No. 14/514,164 filed on Oct. 14, 2014, entitled “Intelligent Continuity of Care Information System and Method”;

U.S. Non-Provisional patent application Ser. No. 14/326,863 filed on Jul. 9, 2014, entitled “Patient Care Surveillance System and Method”;

U.S. Non-Provisional patent application Ser. No. 14/018,514 filed on Sep. 5, 2013, entitled “Clinical Dashboard User Interface System and Method”; and

U.S. Non-Provisional patent application Ser. No. 13/613,980 filed on Sep. 13, 2012 and entitled “Clinical Predictive and Monitoring System and Method.”

### FIELD

The present disclosure relates to the field of electronic medical records, and in particular relates to an automated patient chart review system and method.

### BACKGROUND

There were over 39 million hospital discharges in the United States in 2006. Among Medicare patients, almost 20 percent who are discharged from a hospital are readmitted within 30 days. Unplanned readmissions, at a cost of $17.4 billion, accounted for 17 percent of total hospital payments from Medicare in 2004. Preventing avoidable readmissions has the potential to profoundly improve both the quality-of-life for patients and the financial well-being of healthcare systems. Hospitals are required to address gaps in patient care and transitional care after discharge, which manifest as adverse events (AEs) and readmissions. Failures by hospitals to lower excessive 30-day readmission rates are met with reductions in reimbursement from Medicare & Medicaid.

Readmission is only one of many adverse events and inefficiencies that negatively affect patient outcomes and the financial bottom line. Retrospective chart review is a systematic way to spot issues, improve patient's welfare, help rapidly survey trends, and assist hospitals to lower costs and improve quality.

### BRIEF DESCRIPTION OF THE DRAWINGS

FIG. 1 is a simplified block diagram of an exemplary embodiment of a clinical predictive and monitoring system and method providing patient data to an automated patient chart review system and method according to the present disclosure;

FIG. 2 is a simplified logical block diagram of an exemplary embodiment of a clinical predictive and monitoring system and method providing patient data to an automated patient chart review system and method according to the present disclosure;

FIG. 3 is a diagram illustrative of the volume of patient data for review according to the present disclosure for the example of readmission;

FIG. 4 is a simplified diagram of an exemplary embodiment of an automated patient chart review system and method according to the present disclosure;

FIG. 5 is an exemplary screen shot of an embodiment of an automated patient chart review system and method according to the present disclosure; and

FIGS. 6-9 are additional exemplary screen shots of an embodiment of an automated patient chart review system and method according to the present disclosure.

### DETAILED DESCRIPTION

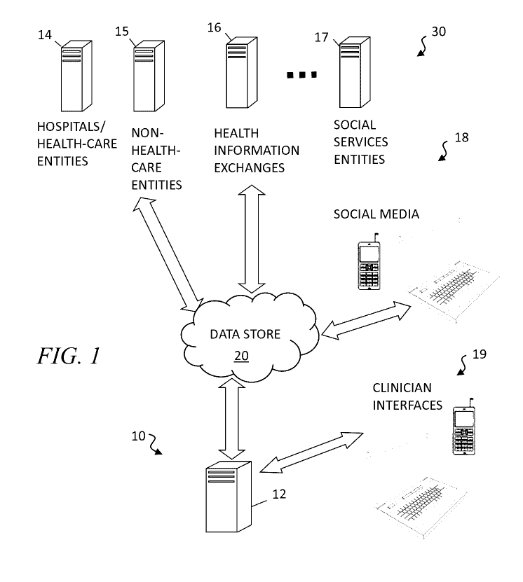

FIG. 1 is a simplified block diagram of an exemplary embodiment of a clinical predictive and monitoring system and method 30 employing a patient protected information de-identification system and method 10 according to the present disclosure. The patient protected information de-identification system 10 includes a computer system 12 adapted to receive a variety of clinical and non-clinical data relating to patients or individuals requiring and receiving care. The variety of data include real-time data streams and historical or stored data from hospitals and healthcare entities 14, non-health care entities 15, health information exchanges 16, and social-to-health information exchanges and social services entities 17, for example. These data may be used to determine a disease risk score for selected patients so that they may receive more target intervention, treatment, and care that are better tailored and customized to their particular condition and needs. The clinical predictive and monitoring system 30 is most suited for identifying particular patients who require intensive inpatient and/or outpatient care to avert serious detrimental effects of certain diseases, to reduce hospital readmission rates, or to identify other adverse events or inefficiencies. It should be noted that the computer system 12 may comprise one or more local or remote computer servers operable to transmit data and communicate via wired and wireless communication links and computer networks.

The data received by the clinical predictive and monitoring system 30 include electronic medical records (EMR) that include both clinical and non-clinical data. The EMR clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing.

The EMR non-clinical data may include, for example, social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social digital data, such as social media activities, phone activity, email activity; and educational level. The non-clinical patient data may further include data entered by the patients, such as data entered or uploaded to a social media website.

Additional sources or devices of EMR data may provide, for example, lab results, medication assignments and changes, EKG results, radiology notes, daily weight readings, and daily blood sugar testing results. These data sources may be from different areas of the hospital, clinics, patient care facilities, laboratories, patient home monitoring devices, among other available clinical or healthcare sources.

As shown in FIG. 1, patient data sources may include non-healthcare entities 15. These are entities or organizations that are not thought of as traditional healthcare providers. These entities 15 may provide non-clinical data that include, for example, gender; marital status; education; community and religious organizational involvement; proximity and number of family or care-giving assistants; address; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources may provide further insightful information about patient lifestyle, such as the number of family members, relationship status, individuals who might help care for a patient, and health and lifestyle preferences that could influence health outcomes.

The clinical predictive and monitoring system 30 may further receive data from health information exchanges (HIE) 16. HIEs are organizations that mobilize healthcare information electronically across organizations within a region, community or hospital system. HIEs are increasingly developed to share clinical and non-clinical patient data between healthcare entities within cities, states, regions, or within umbrella health systems. Data may arise from numerous sources such as hospitals, clinics, consumers, payers, physicians, labs, outpatient pharmacies, ambulatory centers, nursing homes, and state or public health agencies.

A subset of HIEs connect healthcare entities to community organizations that do not specifically provide health services, such as non-governmental charitable organizations, social service agencies, and city agencies. The clinical predictive and monitoring system 30 may receive data from these social services organizations and social-to-health information exchanges 17, which may include, for example, information on daily living skills, availability of transportation to doctor appointments, employment assistance, training, substance abuse rehabilitation, counseling or detoxification, rent and utilities assistance, homeless status and receipt of services, medical follow-up, mental health services, meals and nutrition, food pantry services, housing assistance, temporary shelter, home health visits, domestic violence, appointment adherence, discharge instructions, prescriptions, medication instructions, neighborhood status, and ability to track referrals and appointments.

Another source of data include social media or social network services 18, such as FACEBOOK and GOOGLE+websites. Such sources can provide information such as the number of family members, relationship status, identify individuals who may help care for a patient, and health and lifestyle preferences that may influence health outcomes. These social media data may be received from the websites, with the individual's permission, and some data may come directly from a user's computing device as the user enters status updates, for example.

These non-clinical patient data provides a much more realistic and accurate depiction of the patient's overall holistic healthcare environment. Augmented with such non-clinical patient data, the analysis and predictive modeling performed by the present system to identify patients at high-risk of readmission or disease recurrence become much more robust and accurate.

The clinical predictive and monitoring system 30 is further adapted to receive user preferences and system configuration data from clinicians' computing devices (mobile devices, tablet computers, laptop computers, desktop computers, servers, etc.) 19 in a wired or wireless manner. These computing devices are equipped to display a system dashboard and/or another graphical user interface to present system data and reports configured for an institution (e.g., hospitals and clinics) and individual healthcare providers (e.g., physicians, nurses, and administrators). For example, a clinician (healthcare personnel) may immediately generate a list of patients that have the highest congestive heart failure risk scores, e.g., top n numbers or top x %. The graphical user interface are further adapted to receive the user's (healthcare personnel) input of preferences and configurations, etc. The data may be transmitted, presented, and displayed to the clinician/user in the form of web pages, web-based message, text files, video messages, multimedia messages, text messages, e-mail messages, and in a variety of suitable ways and formats.

As shown in FIG. 1, the clinical predictive and monitoring system 30 may receive and process data streamed real-time, or from historic or batched data from various data sources. Further, the clinical predictive and monitoring system 30 may store the received data in a data store 20 or process the data without storing it first. The real-time and stored data may be in a wide variety of formats according to a variety of protocols, including CCD, XDS, HL7, SSO, HTTPS, EDI, CSV, etc. The data may be encrypted or otherwise secured in a suitable manner. The data may be pulled (polled) by the clinical predictive and monitoring system 30 from the various data sources or the data may be pushed to the system by the data sources. Alternatively or in addition, the data may be received in batch processing according to a predetermined schedule or on-demand. The data store 20 may include one or more local servers, memory, drives, and other suitable storage devices. Alternatively or in addition, the data may be stored in a data center in the cloud.

The computer system 12 may comprise a number of computing devices, including servers, that may be located locally or in a cloud computing farm. The data paths between the computer system 12 and the data store 20 may be encrypted or otherwise protected with a firewall or other security measures and secure transport protocols now known or later developed.

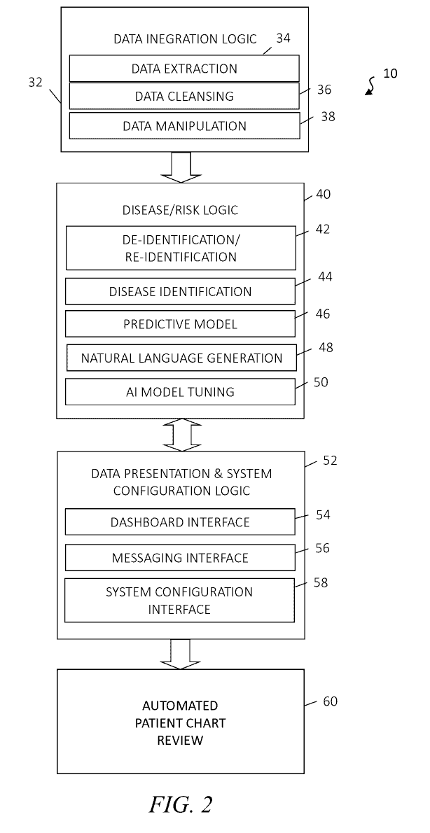

FIG. 2 is a simplified logical block diagram of an exemplary embodiment of a clinical predictive and monitoring system and method 30 that employs the patient protected information de-identification system and method 10. Because the clinical predictive and monitoring system and method 30 receive and extract data from many disparate sources in myriad formats pursuant to different protocols, the incoming data must first undergo a multi-step process before they may be properly analyzed and utilized. The clinical predictive and monitoring system and method 30 includes a data integration logic module 32 that further includes a data extraction process 34, a data cleansing process 36, and a data manipulation process 38. It should be noted that although the data integration logic module 32 is shown to have distinct processes 34-38, these are done for illustrative purposes only and these processes may be performed in parallel, iteratively, and interactively.

The data extraction process 34 may extract clinical and non-clinical data from data sources in real-time or in historical batch files either directly or through the Internet, using various technologies and protocols. Preferably in real-time, the data cleansing process 36 “cleans” or pre-processes the data, putting structured data in a standardized format and preparing unstructured text for natural language processing (NLP) to be performed in the disease/risk logic module 40 described below. The system may also receive “clean” data and convert them into desired formats (e.g., text date field converted to numeric for calculation purposes).

The data manipulation process 38 may analyze the representation of a particular data feed against a meta-data dictionary and determine if a particular data feed should be re-configured or replaced by alternative data feeds. For example, a given hospital EMR may store the concept of “maximum creatinine” in different ways. The data manipulation process 28 may make inferences in order to determine which particular data feed from the EMR would best represent the concept of “creatinine” as defined in the meta-data dictionary and whether a feed would need particular re-configuration to arrive at the maximum value (e.g., select highest value).

The data integration logic module 32 may then pass the pre-processed data to a disease/risk logic module 40. The disease risk logic module 40 is operable to calculate a risk score associated with an identified disease or condition for each patient and identifying those patients who should receive targeted intervention and care. The disease/risk logic module 40 includes a de-identification/re-identification process 42 that is adapted to remove and replace all protected health information (PHI) according to HIPAA standards before the data is shared or transmitted over the Internet. It is also adapted to re-identify the data in the reverse direction. Protected health information that may be removed and added back may include, for example, name, phone number, facsimile number, email address, social security number, medical record number, health plan beneficiary number, account number, certificate or license number, vehicle number, device number, URL, all geographical subdivisions smaller than a state, including street address, city, county, precinct, zip code, and their equivalent geocodes (except for the initial three digits of a zip code, if according to the current publicly available data from the Bureau of the Census), Internet Protocol number, biometric data, and any other unique identifying number, characteristic, or code.

The disease/risk logic module 40 may further include a disease identification process 44. The disease identification process 44 is adapted to identify one or more diseases or conditions of interest for each patient. The disease identification process 44 considers data such as lab orders, lab values, clinical text and narrative notes, and other clinical and historical information to determine the probability that a patient has a particular disease. Additionally, during disease identification, natural language processing is conducted on unstructured clinical and non-clinical data to determine the disease or diseases that the physician believes are prevalent. This process 44 may be performed iteratively over the course of many days to establish a higher confidence in the disease identification as the physician becomes more confident in the diagnosis. New or updated patient data may not support a previously identified disease, and the system would automatically remove the patient from that disease list. The natural language processing combines a rule-based model and a statistically-based learning model.

The disease identification process 44 utilizes a hybrid model of natural language processing, which combines a rule-based model and a statistically-based learning model. During natural language processing, raw unstructured data, for example, physicians' notes and reports, first go through a process called tokenization. The tokenization process divides the text into basic units of information in the form of single words or short phrases by using defined separators such as punctuation marks, spaces, or capitalizations. Using the rule-based model, these basic units of information are identified in a meta-data dictionary and assessed according to predefined rules that determine meaning. Using the statistical-based learning model, the disease identification process 44 quantifies the relationship and frequency of word and phrase patterns and then processes them using statistical algorithms. Using machine learning, the statistical-based learning model develops inferences based on repeated patterns and relationships. The disease identification process 44 performs a number of complex natural language processing functions including text pre-processing, lexical analysis, syntactic parsing, semantic analysis, handling multi-word expression, word sense disambiguation, and other functions.

For example, if a physician's notes include the following: “55 yo m c h/o dm, cri. now with adib rvr, chfexac, and rle cellulitis going to 10 W, tele.” The data integration logic 32 is operable to translate these notes as: “Fifty-five-year-old male with history of diabetes mellitus, chronic renal insufficiency now with atrial fibrillation with rapid ventricular response, congestive heart failure exacerbation and right lower extremity cellulitis going to 10 West and on continuous cardiac monitoring.”

Continuing with the prior example, the disease identification process 44 is adapted to further ascertain the following: 1) the patient is being admitted specifically for atrial fibrillation and congestive heart failure; 2) the atrial fibrillation is severe because rapid ventricular rate is present; 3) the cellulitis is on the right lower extremity; 4) the patient is on continuous cardiac monitoring or telemetry; and 5) the patient appears to have diabetes and chronic renal insufficiency.

The disease/risk logic module 40 further comprises a predictive model process 46 that is adapted to predict the risk of particular diseases or condition of interest according to one or more predictive models. For example, if the hospital desires to determine the level of risk for future readmission for all patients currently admitted with heart failure, the heart failure predictive model may be selected for processing patient data. However, if the hospital desires to determine the risk levels for all internal medicine patients for any cause, an all-cause readmissions predictive model may be used to process the patient data. As another example, if the hospital desires to identify those patients at risk for short-term and long-term diabetic complications, the diabetes predictive model may be used to target those patients. Other predictive models may include HIV readmission, diabetes identification, risk for cardio-pulmonary arrest, kidney disease progression, acute coronary syndrome, pneumonia, cirrhosis, all-cause disease-independent readmission, colon cancer pathway adherence, and others.

Continuing to use the prior example, the predictive model for congestive heart failure may take into account a set of risk factors or variables, including the worst values for laboratory and vital sign variables such as: albumin, total bilirubin, creatine kinase, creatinine, sodium, blood urea nitrogen, partial pressure of carbon dioxide, white blood cell count, troponin-I, glucose, internationalized normalized ratio, brain natriuretic peptide, pH, temperature, pulse, diastolic blood pressure, and systolic blood pressure. Further, non-clinical factors are also considered, for example, the number of home address changes in the prior year, risky health behaviors (e.g., use of illicit drugs or substances), number of emergency room visits in the prior year, history of depression or anxiety, and other factors. The predictive model specifies how to categorize and weight each variable or risk factor, and the method of calculating the predicted probably of readmission or risk score. In this manner, the clinical predictive and monitoring system and method 30 is able to stratify, in real-time, the risk of each patient that arrives at a hospital or another healthcare facility. Therefore, those patients at the highest risks are automatically identified so that targeted intervention and care may be instituted. One output from the disease/risk logic module 40 includes the risk scores of all the patients for particular disease or condition. In addition, the module 40 may rank the patients according to the risk scores, and provide the identities of those patients at the top of the list. For example, the hospital may desire to identify the top 20 patients most at risk for congestive heart failure readmission, and the top 5% of patients most at risk for cardio-pulmonary arrest in the next 24 hours. Other diseases and conditions that may be identified using predictive modeling include, for example, HIV readmission, diabetes identification, kidney disease progression, colorectal cancer continuum screening, meningitis management, acid-base management, anticoagulation management, etc.

The disease/risk logic module 40 may further include a natural language generation module 48. The natural language generation module 48 is adapted to receive the output from the predictive model 46 such as the risk score and risk variables for a patient, and “translate” the data to present the evidence that the patient is at high-risk for that disease or condition. This module 40 thus provides the intervention coordination team additional information that supports why the patient has been identified as high-risk for the particular disease or condition. In this manner, the intervention coordination team may better formulate the targeted inpatient and outpatient intervention and treatment plan to address the patient's specific situation.

The disease/risk logic module 40 may further include an artificial intelligence (AI) model tuning process 50. The artificial intelligence model tuning process 48 utilizes adaptive self-learning capabilities using machine learning technologies. The capacity for self-reconfiguration enables the system and method 30 to be sufficiently flexible and adaptable to detect and incorporate trends or differences in the underlying patient data or population that may affect the predictive accuracy of a given algorithm. The artificial intelligence model tuning process 50 may periodically retrain a selected predictive model for improved accurate outcome to allow for selection of the most accurate statistical methodology, variable count, variable selection, interaction terms, weights, and intercept for a local health system or clinic. The artificial intelligence model tuning process 50 may automatically modify or improve a predictive model in three exemplary ways. First, it may adjust the predictive weights of clinical and non-clinical variables without human supervision. Second, it may adjust the threshold values of specific variables without human supervision. Third, the artificial intelligence model tuning process 50 may, without human supervision, evaluate new variables present in the data feed but not used in the predictive model, which may result in improved accuracy. The artificial intelligence model tuning process 50 may compare the actual observed outcome of the event to the predicted outcome then separately analyze the variables within the model that contributed to the incorrect outcome. It may then re-weigh the variables that contributed to this incorrect outcome, so that in the next reiteration those variables are less likely to contribute to a false prediction. In this manner, the artificial intelligence model tuning process 50 is adapted to reconfigure or adjust the predictive model based on the specific clinical setting or population in which it is applied. Further, no manual reconfiguration or modification of the predictive model is necessary. The artificial intelligence model tuning process 50 may also be useful to scale the predictive model to different health systems, populations, and geographical areas in a rapid timeframe.

As an example of how the artificial intelligence model tuning process 50 functions, the sodium variable coefficients may be periodically reassessed to determine or recognize that the relative weight of an abnormal sodium laboratory result on a new population should be changed from 0.1 to 0.12. Over time, the artificial intelligence model tuning process 38 examines whether thresholds for sodium should be updated. It may determine that in order for the threshold level for an abnormal sodium laboratory result to be predictive for readmission, it should be changed from, for example, 140 to 136 mg/dL. Finally, the artificial intelligence model tuning process 50 is adapted to examine whether the predictor set (the list of variables and variable interactions) should be updated to reflect a change in patient population and clinical practice. For example, the sodium variable may be replaced by the NT-por-BNP protein variable, which was not previously considered by the predictive model.

The results from the disease/risk logic module 40 are provided to the hospital personnel, such as the intervention coordination team, and other caretakers by a data presentation and system configuration logic module 52. The data presentation logic module 52 includes a dashboard interface 54 that is adapted to provide information on the performance of the clinical predictive and monitoring system and method 30. A user (e.g., hospital personnel, administrator, and intervention coordination team) is able to find specific data they seek through simple and clear visual navigation cues, icons, windows, and devices. The interface may further be responsive to audible commands, for example. Because the number of patients a hospital admits each day can be overwhelming, a simple graphical interface that maximizes efficiency and reduce user navigation time is desirable. The visual cues are preferably presented in the context of the problem being evaluated (e.g., readmissions, out-of-ICU, cardiac arrest, diabetic complications, among others).

The dashboard user interface 54 allows interactive requesting of a variety of views, reports and presentations of extracted data and risk score calculations from an operational database within the system. including, for example, summary views of a list of patients in a specific care location; detailed explanation of the components of the various sub-scores; graphical representations of the data for a patient or population over time; comparison of incidence rates of predicted events to the rates of prediction in a specified time frame; summary text clippings, lab trends and risk scores on a particular patient for assistance in dictation or preparation of history and physical reports, daily notes, sign-off continuity of care notes, operative notes, discharge summaries, continuity of care documents to outpatient medical practitioners; order generation to automate the generation of orders authorized by a local care providers healthcare environment and state and national guidelines to be returned to the practitioner's office, outside healthcare provider networks or for return to a hospital or practices electronic medical record; aggregation of the data into frequently used medical formulas to assist in care provision including but not limited to: acid-base calculation, MELD score, Child-Pugh-Turcot score, TIMI risk score, CHADS score, estimated creatinine clearance, Body Surface area, Body Mass Index, adjuvant, neoadjuvant and metastatic cancer survival nomograms, MEWS score, APACHE score, SWIFT score, NIH stroke scale, PORT score, AJCC staging; and publishing of elements of the data on scanned or electronic versions of forms to create automated data forms.

The data presentation and system configuration logic module 52 further includes a messaging interface 56 that is adapted to generate output messaging code in forms such as HL7 messaging, text messaging, e-mail messaging, multimedia messaging, web pages, web portals, REST, XML, computer generated speech, constructed document forms containing graphical, numeric, and text summary of the risk assessment, reminders, and recommended actions. The interventions generated or recommended by the system and method 30 may include: risk score report to the primary physician to highlight risk of readmission for their patients; score report via new data field input into the EMR for use by population surveillance of entire population in hospital, covered entity, accountable care population, or other level of organization within a healthcare providing network; comparison of aggregate risk of readmissions for a single hospital or among hospitals to allow risk-standardized comparisons of hospital readmission rates; automated incorporation of score into discharge summary template, continuity of care document (within providers in the inpatient setting or to outside physician consultants and primary care physicians), HL7 message to facility communication of readmission risk transition to nonhospital physicians; and communicate subcomponents of the aggregate social-environmental score, clinical score and global risk score. These scores would highlight potential strategies to reduce readmissions including: generating optimized medication lists; allowing pharmacies to identify those medication on formulary to reduce out-of-pocket cost and improve outpatient compliance with the pharmacy treatment plan; flagging nutritional education needs; identifying transportation needs; assessing housing instability to identify need for nursing home placement, transitional housing, or Section 8 HHS housing assistance; identifying poor self regulatory behavior for additional follow-up phone calls; identifying poor social network scores leading to recommendation for additional in home RN assessment; flagging high substance abuse score for consultation of rehabilitation counselling for patients with substance abuse issues.

This output may be transmitted wirelessly or via LAN, WAN, the Internet, and delivered to healthcare facilities' electronic medical record stores, user electronic devices (e.g., pager, text messaging program, mobile telephone, tablet computer, mobile computer, laptop computer, desktop computer, and server), health information exchanges, and other data stores, databases, devices, and users. The system and method 30 may automatically generate, transmit, and present information such as high-risk patient lists with risk scores, natural language generated text, reports, recommended actions, alerts, Continuity of Care Documents, flags, appointment reminders, and questionnaires.

The data presentation and system configuration logic module 52 may further include a system configuration interface 58. Local clinical preferences, knowledge, and approaches may be directly provided as input to the predictive models through the system configuration interface 56. This system configuration interface 56 allows the institution or health system to set or reset variable thresholds, predictive weights, and other parameters in the predictive model directly. The system configuration interface 58 preferably includes a graphical user interface designed to minimize user navigation time.

The clinical and non-clinical patient data may be further provided to an automated patient chart review system and method 60. Automated patient chart review system and method 60 are needed to respond to many issues related to manual chart review. As the number of patients admitted to many larger institutions grows, the volume of patient medical records becomes more difficult to manage. The traditional manual review process is difficult to scale to accommodate additional patients, and additional clinical events. Further, traditional manual review processes are generalized and do not focus on specific clinical events. They also do not typically provide consistent and structured survey feedback. A clinical event is defined as a clinical outcome that is of interest to a hospital and/or clinician, such as 30-day readmission, out of ICU, sepsis, and asthma.

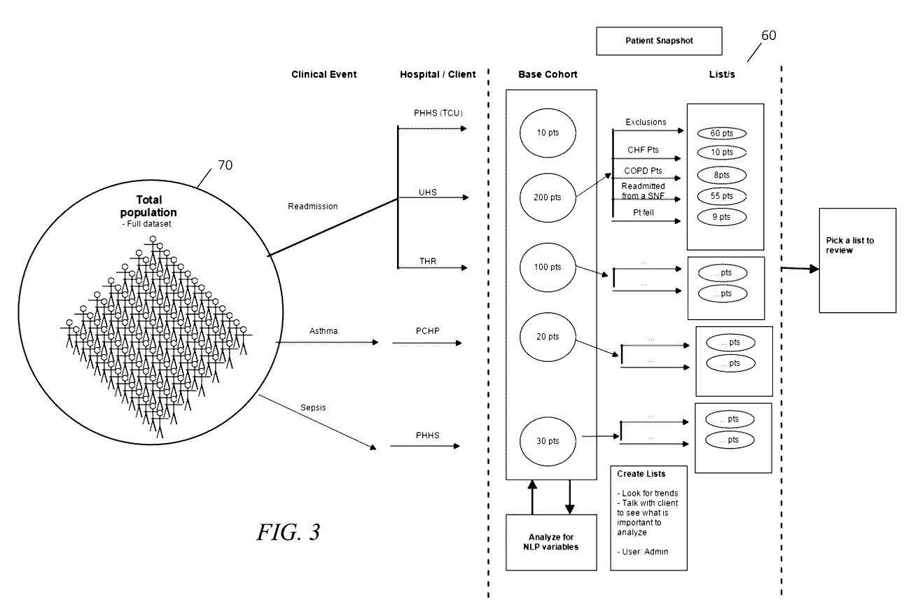

FIG. 3 is a diagram illustrative of the volume of patient data for review according to the present disclosure. FIG. 3 shows that from the total patient population 70, the automated patient chart review system and method 60 may conduct a focused review on specific clinical events or health condition, e.g., 30-day readmission, asthma, and sepsis, or specific patient groups, e.g., all cardiology patients under 65, or specific encounters or notes, e.g., ER visits that led to hospital admissions. Each hospital may conduct its own chart review. Following the UHS example, perhaps 200 patients form the base cohort. Of those 200 patients, the automated patient chart review system and method 60 can further concentrate on, for example, 30-day readmission due to specific diseases or reasons (based on an index admission code assigned to each patient), such as congestive heart failure (CHF), chronic obstructive pulmonary disease (COPD), referral from a skilled nursing facility (SNF), and the patient fell. Of the 200 patients, the automated system and method 60 may automatically identify sixty (60) patients that can be quickly excluded from the review, due to one or more predetermined criteria. For example, one reason to exclude a certain patient from the review may be that the patient is identified as a cancer patient. The automated system and method 60 is operable to automatically generate a specific disease/condition list for review.

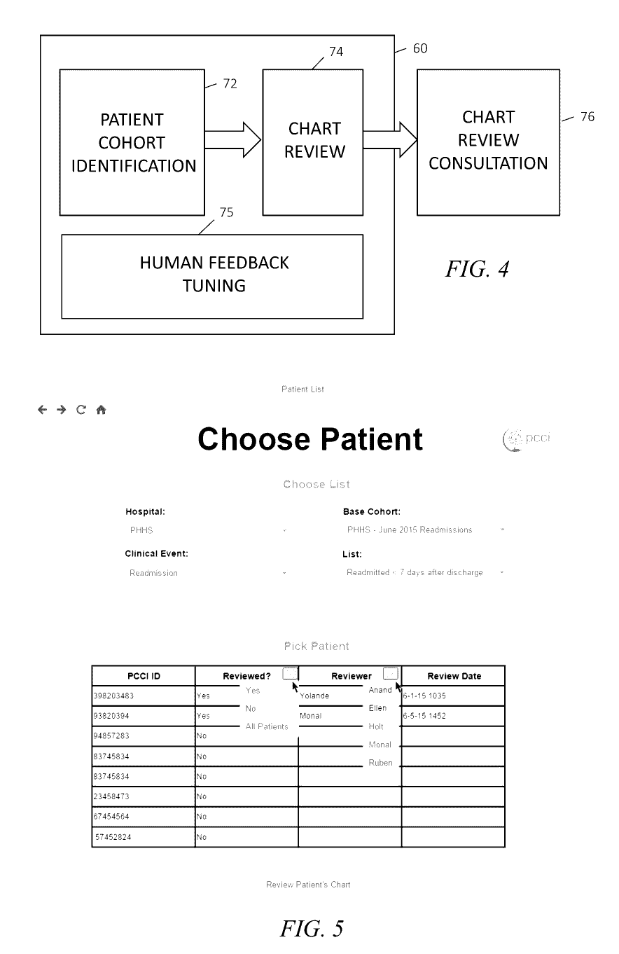

FIG. 4 is a simplified diagram of an exemplary embodiment of an automated patient chart review system and method 60 according to the present disclosure. The automated patient chart review system and method 60 includes two primary components: cohort identification 72 and chart review 74. The cohort identification component 72 is operable to identify and categorize patients, notes, and other information into certain groups in order to enable a focused review. The chart review component 74 is operable to provide an interactive user interface 76 to present patient data and for receiving reviewer comments and feedback. The reviewer feedback may be used to automatically adjust the weights of the plurality of risk variables used in the predictive model used to identify at least one high-risk patient and likely relevant information about the patients.

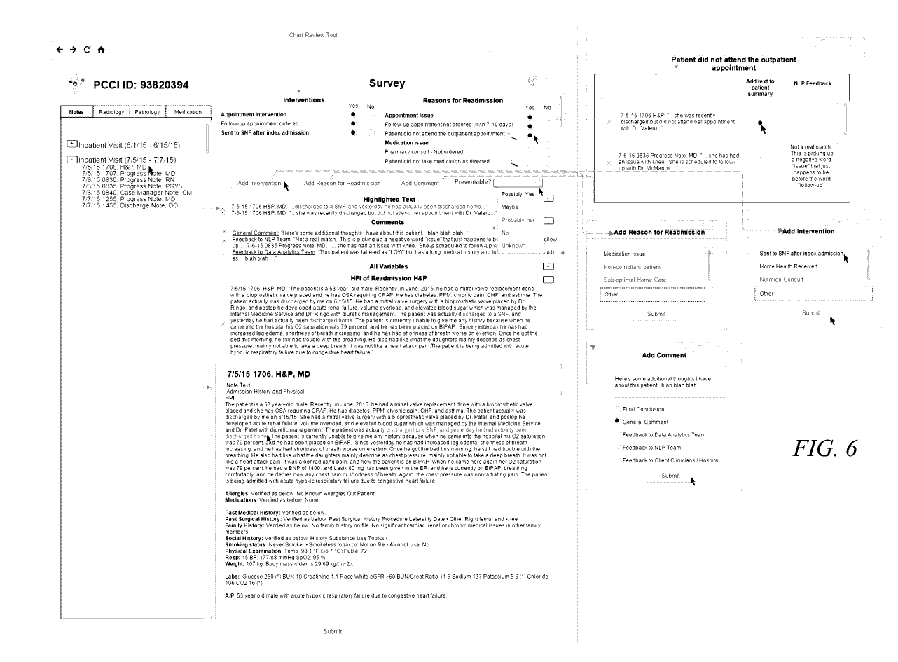

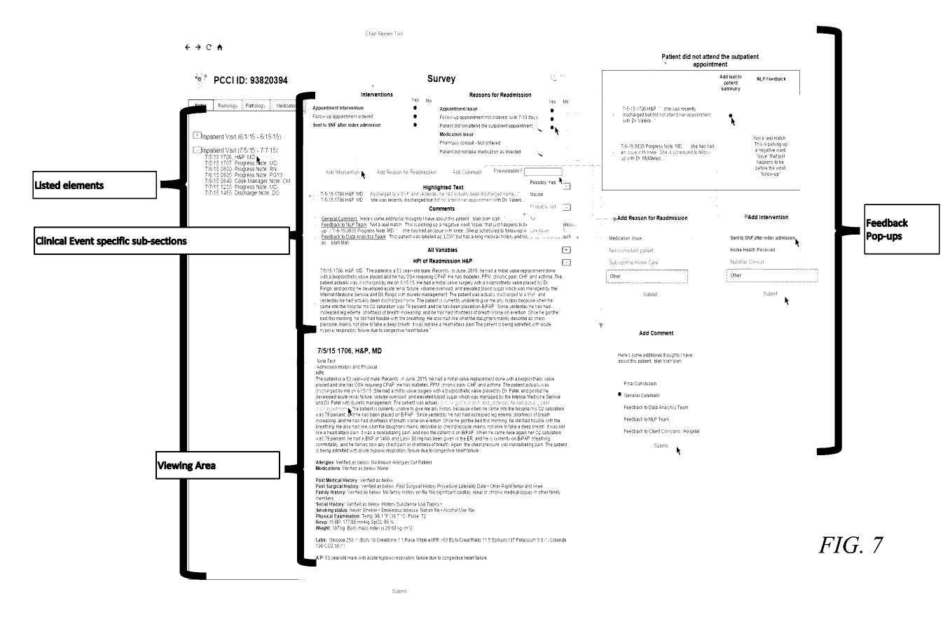

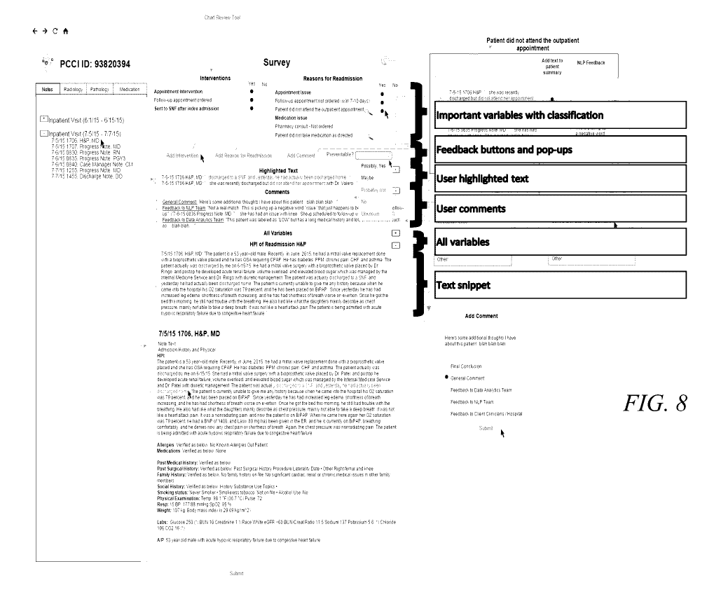

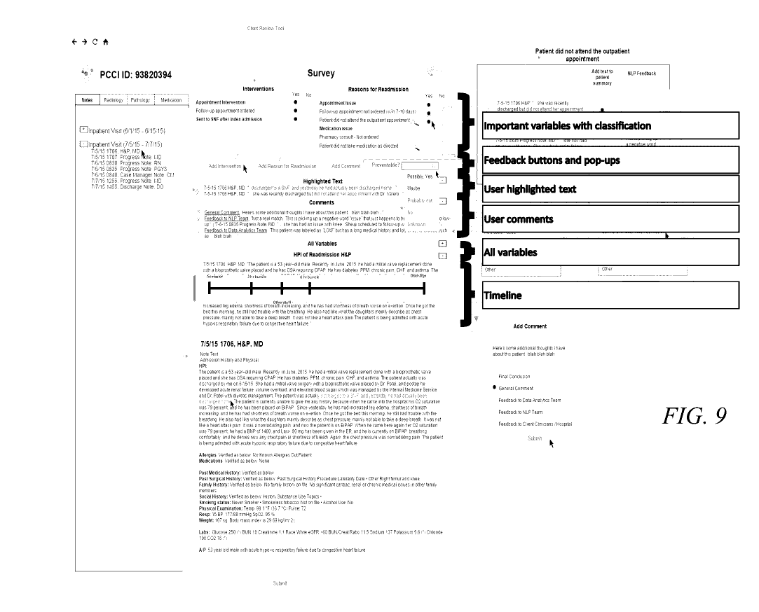

FIG. 5 is an exemplary screen shot of an embodiment of an automated patient chart review system and method 60 according to the present disclosure. The reviewer will first be required to submit credentials (e.g., user name, password, etc.) to log into the system. The reviewer may select a date range, hospital, clinical event, etc. FIG. 5 shows an exemplary initial screen that provides a list of patient cohorts that share a common disease or condition (i.e., clinical event) for review, such as the 30-day readmission example shown. The reviewer may sort the displayed list by whether the patient's chart has been reviewed and who reviewed it. The reviewer can select a particular patient from the list, review the patient's data (details shown in FIGS. 6-9) and document the reviewer name and date.

FIGS. 6-9 are additional exemplary screen shots of an embodiment of an automated patient chart review system and method 60 according to the present disclosure. Referring to FIGS. 6-9, once the reviewer selects a particular record from the cohort list, the user interface provides a detailed screen with a selective view of the electronic medical record (EMR) for the selected patient. These screens are preferably modular so differences between clinical events can be more easily accommodated. Each clinical event may have one or more variables that determine which modules are appropriate for display. The displayed information includes a unique identifier (PCCI ID) and a list of the selected patient's visits or encounters, as well as a list of notes for each encounter (Listed Elements). The Listed Elements may alternatively show lists of pathology reports and radiology reports. The reviewer may select a particular note and see a selected view of the note in a Viewing Area that displays the patient's admission history and physical, including any noted allergies and current medications, as well as pathology report text, and radiology report text.

The automated patient chart review system and method utilizes NLP to identify and highlight important text in the notes and presented separately in the center window. The automatically highlighted text provides focus and is dependent on the type of clinical event. For example, the reviewer for a patient's readmission would be interested in seeing if a patient was sent to an SNF (skilled nursing facility). Additionally, the reviewer may also add highlight to text in this area by identifying/selecting important text in the note. The added highlighting is stored for future views of the note, and the highlighted text is added to the Patient Summary section.

The center window further includes a Clinical Event Specific Sub-Subsection or Patient Summary area that displays a reviewer comment area, all variables area for displaying the entire set of variables from natural language text and structured data specific to the selected clinical event, HPI (Healthcare Performance Improvement) of readmission H&P (history & physical) area, and the full text of the notes associated with the selected inpatient visit. Each sub-section can be expanded or collapsed. Different clinical events may have views with different sub-sections.

At the top of the center window, a plurality of interventions and reasons for readmission are important variables with classification that are automatically noted. For example, for a given clinical event, there are approximately 50 to 100 variables that are collected. However, only a small number of important variables are useful and are automatically presented for chart review. Each variable can be classified as “good” or “bad.” For example, in readmission, there are variables such as interventions that are “good” in regards to the patient's outcome such as “patient got a follow-up appointment.” There are also “bad” variables such as “patient was a no-show for a follow-up appointment.” “Bad” variables can be set apart visually by using a different color text or font. The reviewer can confirm or deny each variable using the radio buttons for “yes” and “no.”

Using Feedback Buttons and Pop-Ups, the reviewer can add intervention(s), reason(s) for readmission, and comment(s). A plurality of canned or standard interventions, comments, and reasons for readmission are displayed for the reviewer's selection to add to ones already determined. The reviewer may also enter free form text instead of using the standard language. The reviewer may add a comment with comment tags: final conclusion, general comment, feedback to hospital, and feedback to clinical prediction and monitoring system and method. The “Preventable?” question includes a pull-down menu with which the reviewer may indicate whether there is anything actionable to improve the patient outcome. The reviewer may also cause a timeline to be displayed that shows important dates associated with the patient's inpatient visits and treatments.

Using the automated patient chart review system and method the reviewer can review the patient charts of false positive and false negative cases to find suggestions to improve the model prediction. A clinician may use the automated patient chart review system and method to review selected patient's charts to help identify and record “good” factors such as clinical interventions and “bad” factors such as causative factors for a particular clinical event. These factors may be later trended across all patients to create a report for a patient chart review consultation service.

Using the automated patient chart review system and method the clinician and hospital may answer the questions of: 1) What happened? For example, the clinician and hospital may ask “how many readmitted patients failed to get a follow-up appointment?” 2) Why did this happen? For example, the clinician and hospital may ask “why did these readmitted patients get a follow-up appointment?” “Was a follow-up appointment not ordered prior to discharge?” “Did the patient cancel the appointment?” “Did the patient not show up for the appointment?” 3) Could this have been prevented? A number of process improvement recommendations may be provided to reduce the occurrence of the identified clinical event based on the queries. One or more reports may be generated to from the chart review process and consultation.

As set forth above, the artificial intelligence model tuning process 50 is configured to automatically adjust the weights of the plurality of risk variables in the predictive model. The artificial intelligence tuning module is configured to automatically adjust the weights of the plurality of risk variables in consideration of the clinical and non-clinical data to identify likely relevant information for a given patient, and to identify at least one high-risk patient. The artificial intelligence tuning module configured to automatically adjust the manner in which how data are displayed.

The system and method 60 further include the patient chart review module 74 (FIG. 3) that is configured to receive and process natural language queries to identify one or more patients based on structured and note data, and to identify relevant information within a patient's medical record. The patient charts review module is also configured to receive and process search queries for specific medical or social concepts to identify one or more patients based on structured and note data, and to identify relevant information within a patient's medical record.

The system and method 60 further include a human feedback tuning module 75 (FIG. 3) configured to automatically adjust the weights of the plurality of risk variables in consideration of that feedback and the clinical and non-clinical data to identify likely relevant information for a given patient, and to identify at least one high-risk patient. The human feedback tuning module is further configured to modify what information is displayed in response to reviewer's input, and modify what information is displayed in response to reviewer's interaction with displayed data.

The features of the present invention which are believed to be novel are set forth below with particularity in the appended claims. However, modifications, variations, and changes to the exemplary embodiments described above will be apparent to those skilled in the art, and the automated patient chart review system and method described herein thus encompasses such modifications, variations, and changes and are not limited to the specific embodiments described herein.

What is claimed is:

1. A computerized method of automated patient chart review, comprising:
receiving a selection of a particular patient;
automatically parsing at least one document of a patient's medical record having structured data and natural language data;
automatically generating a list of variables from the patient's medical record;
automatically generating a list of important variables from the list of variables associated with a specific clinical event from the structured data and natural language data;
being operable to display at least one of the following data:
an identifier of the patient;
a list of the patient's past clinical encounters;
a list of notes associated with each of the patient's past clinical encounters;
a list of the important variables;
a list of all variables;
a selected text portion of the note;
full text of the note;
highlighted text portions of the note; and
being operable to receive input from a reviewer in the form of:
confirmation of the list of important variables;
additional intervention;
additional reason for clinical event;
additional comments; and
additional highlight on a text portion of the note; and
automatically storing the reviewer's input.

2. The computerized method of claim 1, further comprising displaying radiology notes, pathology notes, and medicine notes.

3. The computerized method of claim 1, further comprising displaying social and demographic information.

4. The computerized method of claim 1, further comprising displaying patient claims and payment data.

5. The computerized method of claim 1, further comprising displaying behavioral and mental health data.

6. The computerized method of claim 1, further comprising displaying social digital data, such as social media activities, phone activity, email activity.

7. The computerized method of claim 1, further comprising displaying physical activity data.

8. The computerized method of claim 1, further comprising displaying custom survey assessment data.

9. The computerized method of claim 1, further comprising employing a predictive model including a plurality of weighted risk variables and risk thresholds in consideration of the clinical and non-clinical data to identify at least one high-risk patient.

10. The computerized method of claim 1, further comprising employing a predictive model including a plurality of weighted risk variables and risk thresholds in consideration of the clinical and non-clinical data to identify likely relevant information for a given patient.

11. The computerized method of claim 9, further comprising automatically adjusting the weights of the plurality of risk variables using artificial intelligence in consideration of the clinical and non-clinical data to identify at least one high-risk patient.

12. The computerized method of claim 10, further comprising automatically adjusting the weights of the plurality of risk variables using artificial intelligence in consideration of the clinical and non-clinical data to identify what is likely relevant information for a given patient.

13. The computerized method of claim 11, further comprising receiving reviewer feedback, and automatically adjusting the weights of the plurality of risk variables in consideration of the received reviewer feedback and the clinical and non-clinical data to identify at least one high-risk patient.

14. The computerized method of claim 12, further comprising receiving reviewer feedback, and automatically adjusting the weights of the plurality of risk variables in consideration of the received reviewer feedback and the clinical and non-clinical data to identify likely relevant information for a given patient.

15. The computerized method of claim 1, further comprising receiving input from the reviewer, and modifying displayed information in response to the reviewer's input.

16. The computerized method of claim 1, further comprising monitoring the reviewer actions with respect to the displayed information, and modifying displayed information in response to the reviewer's actions.

17. The computerized method of claim 15, further comprising automatically adjusting the weights and location of what information is displayed on the screen using artificial intelligence.

18. The computerized method of claim 16, further comprising automatically adjusting the weights and location of what information is displayed on the screen using artificial intelligence.

19. The computerized method of claim 1, further comprising receiving data in the patient's medical record in real-time.

20. The computerized method of claim 1, further comprising receiving data in the patient's medical record periodically.

21. The computerized method of claim 1, further comprising receiving and processing natural language queries to identify at least one patient based on structured and note data.

22. The computerized method of claim 1, further comprising receiving and processing natural language queries to identify relevant information within a patient's medical record.

23. The computerized method of claim 1, further comprising receiving and processing search queries for specific medical or social concepts to identify one or more patients based on structured and note data.

24. The computerized method of claim 1, further comprising receiving and processing search queries for specific medical or social concepts to identify relevant information within a patient's information.

25. An automated patient chart review system, comprising:
a patient cohort component operable to:
receive a selection of a particular patient;
automatically parse at least one document of a patient's medical record having structured data and natural language data;
automatically generate a list of variables from the patient's medical record;
automatically generate a list of important variables from the list of variables associated with a specific clinical event from the structured data and natural language data; and
a patient chart review component being operable to display at least one of the following:
an identifier of the patient;
a list of the patient's past clinical encounters;
a list of notes associated with each of the patient's past clinical encounters;
a list of the important variables;
a list of all variables;
a selected text portion of the note;
full text of the note;
highlighted text portions of the note;
and being operable to receive and store input from a reviewer in the form of:
confirmation of the list of important variables;
additional intervention;
additional reason for clinical event;
additional comments; and
additional highlight on a text portion of the note.

26. The automated patient chart review system of claim 25, wherein the patient chart review component is further configured to display radiology notes, pathology notes, and medicine notes.

27. The automated patient chart review system of claim 25, wherein the patient chart review component is further configured to display social and demographic information.

28. The automated patient chart review system of claim 25, wherein the patient chart review component is further configured to display patient claims and payment data.

29. The automated patient chart review system of claim 25, wherein the patient chart review component is further configured to display behavioral and mental health data.

30. The automated patient chart review system of claim 25, wherein the patient chart review component is further configured to display social digital data, such as social media activities, phone activity, email activity.

31. The automated patient chart review system of claim 25, wherein the patient chart review component is further configured to display physical activity data.

32. The automated patient chart review system of claim 25, wherein the patient chart review component is further configured to display custom survey assessment data.

33. The automated patient chart review system of claim 26, further comprising a predictive model including a plurality of weighted risk variables and risk thresholds in consideration of the clinical and non-clinical data to identify at least one high-risk patient.

34. The automated patient chart review system of claim 26, further comprising a predictive model including a plurality of weighted risk variables and risk thresholds in consideration of the clinical and non-clinical data to identify what is likely relevant information for a given patient.

35. The automated patient chart review system of claim 33, further comprising an artificial intelligence tuning module configured to automatically adjust the weights of the plurality of risk variables in consideration of the clinical and non-clinical data to identify at least one high-risk patient.

36. The automated patient chart review system of claim 34, further comprising an artificial intelligence tuning module configured to automatically adjust the weights of the plurality of risk variables in consideration of the clinical and non-clinical data to identify likely relevant information for a given patient.

37. The automated patient chart review system of claim 35, further comprising a human feedback tuning module configured to automatically adjust the weights of the plurality of risk variables in consideration of that feedback and the clinical and non-clinical data to identify at least one high-risk patient.

38. The automated patient chart review system of claim 36, further comprising a human feedback tuning module configured to automatically adjust the weights of the plurality of risk variables in consideration of that feedback and the clinical and non-clinical data to identify likely relevant information for a given patient.

39. The automated patient chart review system of claim 25, further comprising a human feedback tuning module configured to modify what information is displayed in response to reviewer's input.

40. The automated patient chart review system of claim 25, further comprising a human feedback tuning module configured to modify what information is displayed in response to reviewer's interaction with displayed data.

41. The automated patient chart review system of claim 25, further comprising an artificial intelligence tuning module configured to automatically adjust the manner in which how data are displayed.

42. The automated patient chart review system of claim 25, wherein the patient chart review module is configured to receive and process natural language queries to identify one or more patients based on structured and note data.

43. The automated patient chart review system of claim 25, wherein the patient chart review module is configured to receive and process natural language queries to identify relevant information within a patient's medical record.

44. The automated patient chart review system of claim 25, wherein the patient chart review module is configured to receive and process search queries for specific medical or social concepts to identify one or more patients based on structured and note data.

45. The automated patient chart review system of claim 25, wherein the patient chart review module is configured to receive and process search queries for specific medical or social concepts to identify relevant information within a patient's medical record.
